(19) United States
(12) Patent Application Publication
(10) Pub. No.: US 2026/0232918 A1
(43) Pub. Date: Aug. 13, 2026

(54) MEDICAMENT DELIVERY DEVICE AND MEDICAMENT DELIVERY ASSEMBLY

(71) Applicant: SHL Medical AG, Zug (CH)

(72) Inventor: Daniel Scott, Deerfield Beach, FL (US)

(21) Appl. No.: 19/153,789

(22) PCT Filed: Feb. 5, 2024
(86) PCT No.: PCT/EP2024/052764

§ 371 Date: Aug. 5, 2025

Related U.S. Application Data
(60) Provisional application No. 63/444,260, filed on Feb. 9, 2023.

(30) Foreign Application Priority Data
Mar. 10, 2023 (EP) 23161098.1

(51) Int. Cl. A61M 5/32; A61M 5/24; A61M 5/34

(52) U.S. Cl. CPC A61M 5/3204; A61M 5/2422; A61M 5/34; A61M 2005/2474

(57) ABSTRACT

A cap assembly according to the present text for a medicament delivery device comprises an outer cap and a needle cap. The needle cap has a fastening thread for the needle cap to be fastened to a retaining structure of the medicament delivery device. The outer cap has a cap wall extending from a proximal end towards distally. The cap wall ends in a distal rim A plurality of cut-outs extend from the distal rim towards proximally, with at least one arm section therebetween. The at least one arm section is capable of being flexed inwardly in a resilient manner and has, on an inner side, an outer coupling structure The cap assembly further comprises an inner coupling structure. The outer cap is rotationally de-coupled from the needle cap when the arm arrangement is in an initial, un-flexed state. A rotational coupling between the outer cap and the needle cap requires at least one of the arm sections to be flexed inwardly for the outer coupling structure to engage with the inner coupling structure Thereby, unscrewing the needle cap from the retaining structure requires the outer cap to be turned, with the arm section or at least one of the arm sections flexed inwardly.

### TECHNICAL FIELD

The invention is in the field of medicament delivery devices. More in particular, it relates to an automatic medicament delivery device being safe and easy to handle.

### BACKGROUND

Among the medicament delivery devices for self-administration, there are medicament delivery devices that have a needle pre-attached thereto, for perforating, immediately prior to use, a septum of the medicament container accommodated in the medicament delivery device, and for delivering the medicament in the medicament container to a dose delivery site. Such medicament delivery devices may comprise a cap assembly with a needle cap that protects the needle before use. For use, the needle cap has to be removed.

WO 2013/089616 discloses an example of a medicament delivery device of this kind. The cap assembly comprises a needle hub, whereby the needle acts to pierce the septum upon removal of the needle cap. As a safety feature, the cap assembly comprises an outer cap in addition to the needle cap, with a cap clutch between the outer cap and the needle cap. For rotationally coupling the outer cap to the needle cap, a dose knob arranged at the distal of the medicament delivery device end has to be turned for unlocking. This turning releases a needle guard and allows it to travel in an axial direction to move a locking member of the clutch so as to rotationally couple the outer cap to the needle cap. Only then, by turning the outer cap, may the outer cap and the needle cap be removed. Also, by turning the outer cap together with the needle cap, the needle is caused to pierce the septum. The cap clutch is biased to prevent the locking member from getting, prior to the turning of the dose knob and without the user's intention, into the position in which it rotationally couples the inner and outer caps with respect to each other.

This solution is safe and reliable. However, the coupling of the inner and outer caps just requires a turning of the dose knob, and on the one hand, for applications that require a high safety, it would be desirable to have a cap assembly that includes further safety feature. On the other hand, for other applications there is still room for improvement in terms of reduced complexity.

### SUMMARY

Accordingly, it is an object of the present invention to provide a medicament delivery device that overcomes at least some of the drawbacks of the prior art. Especially, the medicament delivery device should be advantageous in terms of safety, reliability, ease of handling and/or complexity.

This object is achieved by the invention as defined in the appended claims, to which reference should now be made.

In the present disclosure, when the term “distal direction” is used, this refers to the direction pointing away from the dose delivery site during use of the medicament delivery device. When the term “distal part/end” is used, this refers to the part/end of the delivery device, or the parts/ends of the members thereof, which during use of the medicament delivery device is/are located furthest away from the dose delivery site. Correspondingly, when the term “proximal direction” is used, this refers to the direction pointing towards the dose delivery site during use of the medicament delivery device. When the term “proximal part/end” is used, this refers to the part/end of the delivery device, or the parts/ends of the members thereof, which during use of the medicament delivery device is/are located closest to the dose delivery site.

Further, the terms “longitudinal”, “longitudinally”, “axially” and “axial” refer to a direction extending from the proximal end to the distal end and along the device or components thereof, typically in the direction of the longest extension of the device and/or component.

Similarly, the terms “transverse”, “transversal” and “transversally” refer to a direction generally perpendicular to the longitudinal direction.

Generally, all terms used in the claims are to be interpreted according to their ordinary meaning in the technical field, unless explicitly defined otherwise herein. All references to a/an/the element, apparatus, member, component, means, etc. are to be interpreted openly as referring to at least one instance of the element, apparatus, member component, means, etc., unless explicitly stated otherwise.

According to an aspect of the present invention, a cap assembly for a medicament delivery device is provided. In this, the medicament delivery device has a proximal end and a distal end and defines an axis extending proximodistally. For example, the medicament delivery device may be of the kind perceived as ‘pen’ by the user, for a needle arranged proximally and extending along the axis to deliver the medicament. In medicament delivery devices of this kind, for expelling the medicament, often an expelling unit comprises a plunger element that is moved, for example by a spring force, towards proximally to act on a medicament container contained in the medicament delivery device.

The cap assembly comprises an outer cap and a needle cap, with the needle cap having a fastening structure for the needle cap to be fastened to a retaining structure of the medicament delivery device. The fastening structure cooperates with the retaining structure so that a removal of the needle cap from the retaining structure requires a rotation of the needle cap relative to the retaining structure in an opening direction. Examples of a fastening structure include a fastening thread or a bayonet coupling structure.

Such retaining structure may, according to a first option, belong to a retainer of the cap assembly. The cap assembly may be a pre-assembled unit, which unit may be secured to a medicament container sub-assembly-which is equipped to accommodate the medicament container and may essentially consist of the elements of the medicament delivery device which do not belong to the cap assembly-using an engagement structure of the retainer for securing, for example after placing the medicament container relative to the medicament container sub-assembly.

Alternatively, according to a second option, the retaining structure may be a retaining structure of the medicament container sub-assembly itself, i.e., a part of the medicament container sub-assembly may have the retaining structure, for example retaining thread, engaging with the fastening structure, for example fastening thread, of the needle cap.

The outer cap has a cap wall extending from a proximal end towards distally, around the axis, coaxially with the needle cap, with a distal rim.

A plurality of cut-outs extends from the distal rim towards proximally, so that therebetween there is an arm arrangement with one arm section or more arm sections. The arm section(s) is/are capable of being resiliently flexed radially-inwardly and has/have, on an inner side, an outer coupling structure.

The cap assembly further comprises an inner coupling structure rotationally coupled to the needle cap. The outer cap is rotationally de-coupled from the needle cap when the arm arrangement is in an initial, un-flexed state.

Rotational coupling between the outer cap and the needle cap requires the arm section or at least one of the arm sections to be flexed inwardly for the outer coupling structure to engage with the inner coupling structure.

Thus, the inward flexing of the arm section/at least one of the arm sections is a necessary condition for the user to be able to rotate the needle cap by rotating the outer cap.

In the present text, two items are “rotationally coupled” if both items rotate together around the axis when one of the items is caused to rotate-thus, as long as the rotational coupling exists, the orientation of one of the items around the axis also defines the rotation of the other one around the axis. A rotational coupling may be caused if one item is integral with the other item, or if it engages with the other item in the rotational direction, whereas relative movements between the two items in an axial direction may (but need not) be possible.

The number of the cut-outs is at least two, while a configuration with four cut-outs arranged regularly around the circumference is often beneficial because this leaves two pairs of opposing arms so that squeezing is particularly ergonomic. In configurations with a plurality of the arms capable of being flexed inwardly, it may be sufficient if one of the arms has the outer coupling structure. Often, it is beneficial if all arms do so.

According to a first option, the inward flexing of the arm section/at least one of the arm sections in addition to being a necessary condition is also a sufficient condition. For rotating the needle cap, the user thus just has to squeeze and rotate the outer cap. This first option allows to leave away other, more complicated safety features, whereby if configured according to this first option, the present invention reduces complexity.

According to a second option, the cap assembly requires a further action, in addition to the inward flexing of the arm section/at least one of the arm sections. Such further action may for example comprise activation of a clutch mechanism, substantially as described in the prior art, for example in WO 2013/089616. If configured according to this second option, the present invention adds safety without adding complexity, since no further part is required.

In both, embodiments of the first option and in embodiments of the second option, the cap assembly may further comprise a cap clutch. The cap clutch is an element separate from both, the outer cap and the needle cap, but that is rotationally couplable to both, the needle cap and the outer cap. For example, the cap clutch may be constantly rotationally coupled to one of the caps (for example the needle cap) whereas rotational coupling to the other cap (for example the outer cap) requires the arm section(s) to be flexed inwardly and, in the second option, is subject to a further condition. Such further condition may in this for example be that a coupling portion of the clutch is in a desired axial position into which it is brought by a user induced further action. The cap clutch may comprise the inner coupling structure.

Thus, in embodiments with a cap clutch constantly rotationally coupled with the needle cap, the inner coupling structure is rotationally coupled with the needle cap in that the cap clutch, to which the inner coupling structure belongs in these embodiments, is engaged with the needle cap in the rotational direction. Especially, the needle cap may comprise a section with a non-round outer cross section, and the cap clutch may comprise an inner contour cooperating therewith for the rotational coupling. For example, the section of the needle cap may have a cross section of a polygon shape, with the inner contour of the cap clutch having an adapted contour with the same polygon shape. In addition or as an alternative, the section may have axial grooves into which inward protrusions of the cap clutch engage, and/or may have axially running ridges that engage into cut-outs of the cap clutch, etc.

As an alternative to the cap assembly comprising a cap clutch, the inner coupling structure may be a structure of the needle cap itself. This may especially (but not only) be an option in embodiments according to the first option, in which the inward flexing of the arm section/at least one of the arm sections is also a sufficient condition for a rotational coupling between the needle cap and the outer cap.

Thus, according to this alternative, the inner coupling structure is rotationally coupled to the needle cap by being integral with it.

The inner coupling structure and/or the outer coupling structure(s) may be toothed structures. In embodiments, the inner and outer coupling structures may together form a ratchet, whereby rotation in the opening (for example unscrewing) direction can be transferred from the outer cap to the needle cap if the outer cap is squeezed, whereas rotations in the other direction (the opening direction, referring to the retaining structure engaging with the fastening structure) is only possible if the user applies undue squeezing forces.

In a group of embodiments, the cap assembly further comprises a needle hub that is arranged to be moved, by rotating the needle cap in the opening direction, towards distally. The needle hub in this may hold a needle attached to it. When the needle hub is moved towards distally, the needle pierces a septum of the medicament container. To this end, the retainer may comprise a second retainer thread, and the needle hub may comprise a hub thread engaging with the second retainer thread and being rotationally coupled to the needle cap.

In addition to concerning the cap assembly, the present invention also concerns a medicament delivery device that has such a cap assembly, and a medicament delivery assembly with the medicament delivery device and a medicament container.

In addition to the cap assembly, the medicament delivery device has a housing and an expelling unit for expelling a medicament from a medicament container seated within the housing. The expelling unit may comprise a plunger element equipped to be driven in an axial direction while acting on the medicament container to expel the medicament.

The housing, for example in addition to comprising an outer housing sleeve, may comprise a container housing, to which the retainer of the cap assembly is connectable.

### BRIEF DESCRIPTION OF THE DRAWINGS

Embodiments of the present disclosure will now be described by way of example only and with reference to the following accompanying drawings.

The drawings show:

FIG. 1 a view of a medicament delivery assembly in an initial state;

FIG. 2 elements of a cap assembly of the medicament deliver assembly in an exploded view;

FIG. 3 the outer cap of the cap assembly, with the cap clutch inserted;

FIG. 4 the outer cap of the cap assembly;

FIG. 5 the cap clutch;

FIG. 6 a variant of the needle cap of the cap assembly; and

FIG. 7 a variant of the medicament delivery assembly of FIG. 6, again in an initial state.

### DETAILED DESCRIPTION

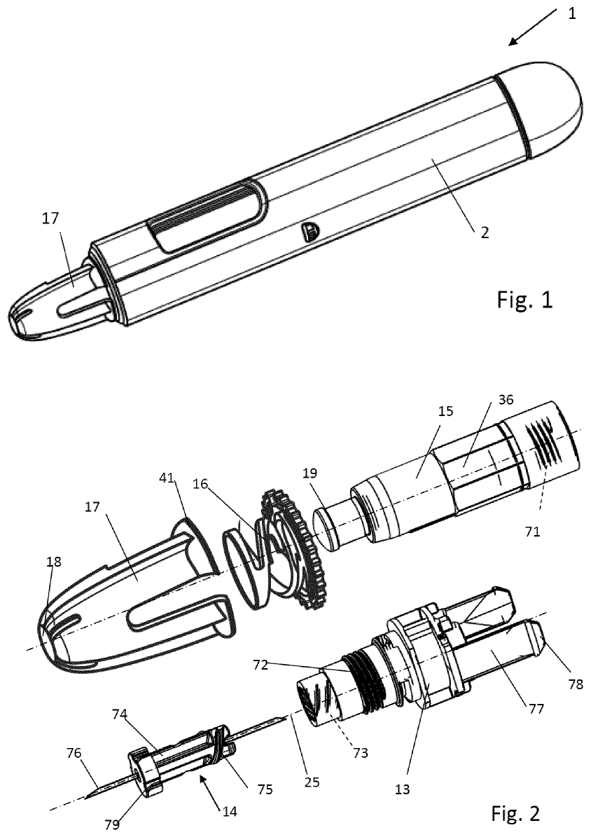

The embodiment of a medicament delivery assembly 1 shown in FIG. 1 comprises a medicament delivery device and a medicament container. The medicament delivery device comprises a housing that has a housing sleeve 2. Within the housing sleeve 2, a container housing for the medicament container is arranged.

Proximally of the medicament container, the medicament delivery device has a cap assembly, the elements of which are shown in an exploded view in FIG. 2. The cap assembly is configured to be attached, directly or indirectly, to a housing of the medicament delivery device, for example a container housing thereof, after the medicament container has been inserted, substantially as described in WO 2013/089616.

The cap assembly comprises a retainer 13 with two distally extending retainer arms 77 with outward protrusions 78 at their free ends. The outward protrusions 78 engage into cut-outs of the container housing or other structure of the medicament delivery device. The cap assembly further comprises a needle assembly 14 housed in the needle cap 15. The needle cap 15 has a cap inner thread 71 (shown only schematically in FIG. 2) that engages with a retainer outer thread 72 arranged on a proximal portion of the retainer, which proximal portion reaches into the needle cap 15 from distally. The retainer further has a retainer inner thread 73 (again shown schematically in FIG. 2). The needle assembly 14 has a needle hub 74 to which the needle 76 is fixedly connected. The needle hub 74 has a hub outer thread 75 engaging with the retainer inner thread 73. The needle hub 74 is rotationally coupled to the needle cap 15 by inwardly extending ribs (not shown in FIG. 2) of the needle cap 15 engaging into axial grooves 79 of the needle hub 74. However, the needle hub 74 is axially movable relative to the needle cap 15.

Therefore, when the needle cap 15 is rotated in the opening (unscrewing) direction, the needle hub 74 rotates with the needle cap 15. Because of the hub outer thread 75 engaging with the retainer inner thread 73, this rotation causes an axial movement towards distally, whereby the needle 76 is caused to pierce a septum of the medicament container, which medicament container is accommodated in the housing of the medicament delivery device and is arranged proximally to the retainer 13.

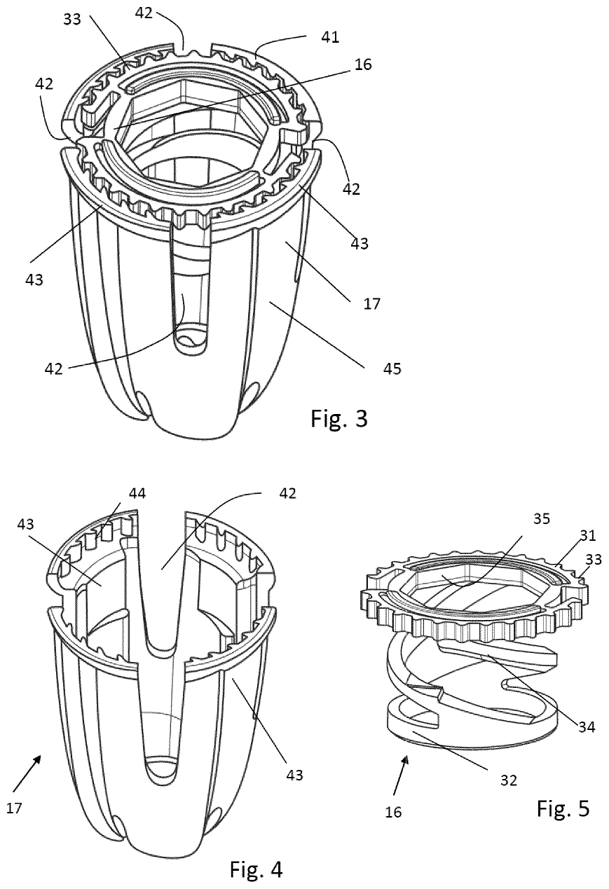

For rotationally coupling the needle cap 15 to an outer cap 17 so that it can be rotated by a user by rotating the outer cap 17, the cap assembly further has a cap clutch 16, which is also illustrated, together with the outer cap 17, in FIG. 3. FIG. 4 shows the outer cap, and FIG. 5 depicts the cap clutch 16.

The cap clutch 16 has a coupling portion 31 with an inner coupling structure 33 a base ring 32, and an intermediate portion 34. The cap clutch 16 further has a rotation coupling structure 35 to be rotationally coupled to the needle cap 15. More in particular, the needle cap has a section with a non-round cross section on which the cap clutch 16 sits. In the depicted embodiment, this section and the rotation coupling structure 35 have an octagonal cross section. Other cross sections, such as hexagonal, rectangular, round with axial ridges or notches, etc. would be possible, too.

The outer cap 17 is generally cup-shaped with cap wall 45 distally ending in a distal rim 41 around the distal mouth, and with a proximal hole 18 for a proximal section 19 of the needle cap 15 to extend through. The proximal section 19 has a small proximal outward bulge securing the outer cap against escaping towards proximally relative to the needle cap 15.

The cap wall 45 of the outer cap 17 comprises a plurality of cut-outs 42—four cut-outs in the depicted embodiment—extending from the distal rim 41 towards proximally. The arm sections 43 that arise due to the cut-outs 42 are capable of being flexed radially inwardly. The distal ends of the arm sections each have an outer coupling structure 44 capable of engaging with the inner coupling structure 33 to rotationally couple the cap clutch 16 with the outer cap 17 when the corresponding arm section 43 is flexed inwardly and the inner coupling structure 33 and the outer coupling structure 44 are located at axial positions that correspond to each other. Thus, the outer cap 17 may be rotationally coupled to the needle cap 15 by squeezing the outer cap 17 so that the arm sections 43 are manually pressed inwardly. In this state, with the outer cap 17 rotationally coupled to the needle cap 15, the user can twist the outer cap 17 and the needle cap 15 together while she/he squeezes the outer cap 17.

In the depicted embodiment, at least one of the inner coupling structure 33 and the outer coupling structures 44—is/are asymmetrical with respect to the circumferential direction, i.e., the inner coupling structure 33 and the outer coupling structures together form a ratchet, whereby rotation in one direction (counterclockwise if seen from the proximal end of the outer cap 17 in the depicted embodiment) can be coupled into the cap clutch 16, whereas rotation into the other direction can only if a strong pressing force is applied on opposing arm sections. In the depicted embodiment, the outer coupling structures 44 are asymmetrical.

The fact that the outer cap is only rotationally coupled to the cap clutch—and thereby to the needle cap—if the outer cap is squeezed is a safety feature preventing the needle cap from being removed (thus unscrewed in the depicted embodiment) unintentionally.

According to the hereinbefore discussed first option, the coupling portion 31 of the cap clutch 16 is permanently located at an axial position that corresponds to the axial position of the outer coupling structures 44, as illustrated in FIGS. 1 and 3. In accordance with this first option, the safety feature is present instead of a mechanism of the kind taught in WO 2013/089616 in which the coupling portion 31 has to be moved, especially by a needle guard front, against a biasing force for being brought into the axial position of the outer coupling structures 44. Therefore, if the cap assembly is configured in accordance with this first option, the rest of the medicament delivery device can be constructed substantially simpler.

If made according to this first option, also the cap assembly itself can be modified to be simplified even further. For example, the cap clutch sitting on the needle cap 15 can be simplified to consist of the coupling portion 31 only, in contrast to the depicted embodiment. Thus, in this embodiment, the cap clutch does not have any base ring 32 or intermediate portion 34.

Even more, the coupling portion can be one-piece with the needle cap, thus making the inner coupling structure 33 features of the needle cap 15 itself.

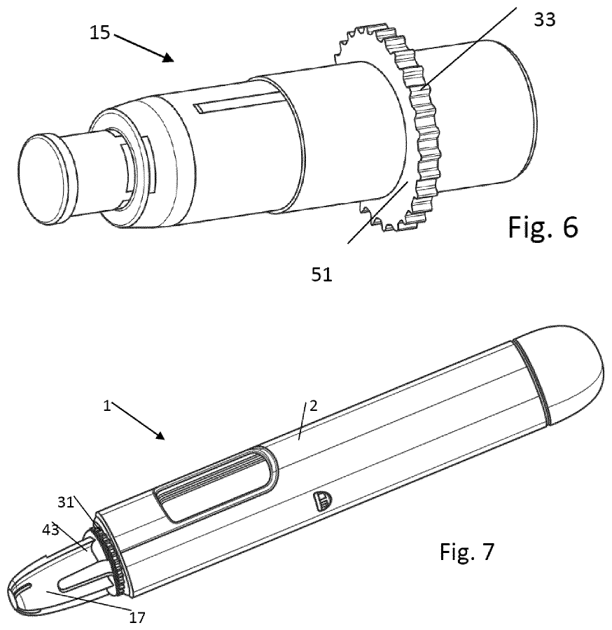

This is schematically illustrated in FIG. 6 showing a variant of the needle cap with comprising an outward extension 51 having the inner coupling structure 33.

As an alternative to the first option, according to the second option, the safety feature may be an additional safety feature, in addition to safety feature as known from the prior art, for example from WO 2013/089616. FIG. 7 illustrates this possibility by showing a medicament delivery assembly 1 that has a cap assembly of the kind shown in FIG. 2, in which, however, the outer cap 17 and the cap clutch are dimensioned so that the coupling portion 31 of the cap clutch is, in an initial state, axially moved relative to the outer coupling structures of the outer cap 17. The intermediate portion 34 acts to urge the coupling portion 31 out of the outer cap 17 by a resilient biasing force caused by the construction of the intermediate portion. More in particular, the coupling portion of the cap clutch in the initial state is distally of the distal rim of the outer cap 17. Only after the mechanism for displacing the coupling portion 31 towards proximally, into the outer cap 17, has been activated, may the outer cap 17 be rotationally coupled to the needle cap, which action again requires squeezing of the outer cap to flex the arm section towards radially-inwardly.

Embodiments in accordance with this second option feature the advantage of being even safer than corresponding prior art cap assemblies and medicament delivery devices, without adding substantial complexity to the construction of the cap assembly/medicament delivery device.

The drug delivery devices described herein can be used for the treatment and/or prophylaxis of one or more of many different types of disorders.

Exemplary disorders include, but are not limited to: rheumatoid arthritis, inflammatory bowel diseases (e.g. Crohn's disease and ulcerative colitis), hypercholesterolaemia, diabetes (e.g. type 2 diabetes), psoriasis, migraines, multiple sclerosis, anaemia, lupus, atopic dermatitis, asthma, nasal polyps, acute hypoglycaemia, obesity, anaphylaxis and allergies. Exemplary types of drugs that could be included in the medicament delivery devices described herein include, but are not limited to, antibodies, proteins, fusion proteins, peptibodies, polypeptides, pegylated proteins, protein fragments, protein analogues, protein variants, protein precursors, and/or protein derivatives.

Exemplary drugs that could be included in the drug delivery devices described herein include, but are not limited to (with non-limiting examples of relevant disorders in brackets): etanercept (rheumatoid arthritis, inflammatory bowel diseases (e.g. Crohn's disease and ulcerative colitis)), evolocumab (hypercholesterolaemia), exenatide (type 2 diabetes), secukinumab (psoriasis), erenumab (migraines), alirocumab (rheumatoid arthritis), methotrexate (amethopterin) (rheumatoid arthritis), tocilizumab (rheumatoid arthritis), interferon beta-1a (multiple sclerosis), sumatriptan (migraines), adalimumab (rheumatoid arthritis), darbepoetin alfa (anaemia), belimumab (lupus), peginterferon beta-1a′ (multiple sclerosis), sarilumab (rheumatoid arthritis), semaglutide (type 2 diabetes, obesity), dupilumab (atopic dermatis, asthma, nasal polyps, allergies), glucagon (acute hypoglycaemia), epinephrine (anaphylaxis), insulin (diabetes), atropine and vedolizumab (inflammatory bowel diseases (e.g. Crohn's disease and ulcerative colitis)). Pharmaceutical formulations including, but not limited to, any drug described herein are also contemplated for use in the drug delivery devices described herein, for example pharmaceutical formulations comprising a drug as listed herein (or a pharmaceutically acceptable salt of the drug) and a pharmaceutically acceptable carrier. Pharmaceutical formulations comprising a drug as listed herein (or a pharmaceutically acceptable salt of the drug) may include one or more other active ingredients, or may be the only active ingredient present.

Various modifications to the embodiments described are possible and will occur to those skilled in the art without departing from the invention which is defined by the following claims.

1-14. (canceled)

15. A cap assembly for a medicament delivery device, the medicament delivery device having a proximal end and a distal end and defining an axis extending proximodistally, the cap assembly comprising an outer cap and a needle cap,
wherein the needle cap has a fastening structure for the needle cap to be fastened to a retaining structure of the medicament delivery device, the fastening structure cooperating with the retaining structure so that a removal of the needle cap from the retaining structure requires a rotation of the needle cap relative to the retaining structure in an opening direction,
wherein the outer cap has a cap wall extending from a proximal end towards distally, around the axis, coaxially with the needle cap, with a distal rim, a plurality of cut-outs extending from the distal rim towards proximally, and therebetween an arm arrangement with at least one arm section,
wherein the at least one arm section is capable of being resiliently flexed radially-inwardly and has, on an inner side, an outer coupling structure,
wherein the cap assembly further comprises an inner coupling structure rotationally coupled to the needle cap,
wherein the outer cap is rotationally de-coupled from the needle cap when the arm arrangement is in an initial, un-flexed state,
wherein a rotational coupling between the outer cap and the needle cap requires the arm section or at least one of the arm sections to be flexed inwardly for the outer coupling structure to engage with the inner coupling structure, and
wherein removal of the needle cap from the retaining structure requires the outer cap to be rotated in the opening direction, with the arm section or at least one of the arm sections flexed inwardly.

16. The cap assembly according to claim 15, wherein the inner coupling structure and the outer coupling structure are toothed structures.

17. The cap assembly according to claim 15, further comprising a retainer, the retainer comprising the retaining structure.

18. The cap assembly according to claim 17, wherein the fastening structure is a fastening thread, and wherein the retaining structure is a first retainer thread cooperating with the fastening thread.

19. The cap assembly according to claim 17, wherein the retainer comprises a second retainer thread, wherein the cap assembly further comprises a needle hub and a needle attached to the needle hub, the needle hub comprising a hub thread engaging with the second retainer thread and being rotationally coupled to the needle cap, whereby rotation of the needle cap causes the needle hub with the needle to be subject to a movement in a distal direction for piercing a septum of a medicament container accommodated, proximally of the retainer, in the medicament delivery device.

20. The cap assembly according to claim 15, further comprising a cap clutch rotationally coupled to the needle cap, the cap clutch comprising the inner coupling structure.

21. The cap assembly according to claim 20, wherein the cap clutch is rotationally coupled to the needle cap by having a non-round inner contour engaging with a non-round cross section of a section of the needle cap.

22. The cap assembly according to claim 20, wherein the cap clutch comprises a coupling portion with the inner coupling structure, and wherein the coupling portion is movable relative to the needle cap in axial directions, between a coupling position, in which the outer coupling structure engages the inner coupling structure when the arm section or at least one of the arm sections is flexed radially-inwardly, and a decoupled position in which no coupling between the outer coupling structure and the inner coupling structure is possible.

23. The cap assembly according to claim 15, wherein the inner coupling structure is an integral structure of the needle cap.

24. The cap assembly according to claim 15, wherein the inner coupling structure and the outer coupling structure together form a ratchet.

25. The cap assembly according to claim 15, wherein the outer cap comprises four of the cut-outs and four of the arm sections with the outer coupling structure.

26. A medicament delivery device, comprising a housing having a proximal end and a distal end and defining an axis extending proximodistally, and an expelling unit for expelling a medicament from a medicament container seated within the housing, the expelling unit comprising plunger element equipped to be moved in the proximal direction relative to the medicament container while acting on the medicament container to expel the medicament, the medicament delivery device further comprising a cap assembly according to claim 15.

27. The medicament delivery device according to claim 26, wherein the housing comprises a container housing accommodating the container, wherein the retainer is connectable to the container housing.

28. A medicament delivery assembly, comprising the medicament delivery device according to claim 26, and further comprising the medicament container assembled with the medicament delivery device.
